(19) United States
(12) Patent Application Publication
(10) Pub. No.: US 2011/0247120 A1
(43) Pub. Date: Oct. 13, 2011

(54) EYEWEAR CONNECTOR AND KIT FOR A HAT

(76) Inventor: Ronnie R. Knoedler, St. Louis, MO (US)

(21) Appl. No.: 12/755,914

(22) Filed: Apr. 7, 2010

(51) Int. Cl. A61F 9/00; B65D 71/00

(52) U.S. Cl. 2/10; 206/223

(57) ABSTRACT

An eyewear connector and a kit for a hat are disclosed, the connector comprising an upper portion and a lower portion and a spine there between. The upper end of the spine has an apex and the lower end of the spine has a nadir. The upper portion of the connector is configured to releasably attach the connector to and suspend the connector from a sweatband positioned horizontally within a piece of headwear. The lower portion of the connector is configured to releasably hold a first arm of a piece of eyewear in a substantially horizontal orientation relative to the sweatband when the first arm is held between the lower portion and the spine and when the upper portion is suspended from the sweatband. The kit comprises two connectors and a matching lens component. The connectors and the lens component may be configured to store within a hat or other headwear.

### CROSS-REFERENCE TO RELATED APPLICATIONS

Not Applicable.

### STATEMENT REGARDING FEDERALLY SPONSORED RESEARCH

Not Applicable.

### BACKGROUND OF THE INVENTION

When wearing headwear, such as for example a hat, it is often desirable to wear eyewear such as eyeglasses or sunglasses at the same time. Traditionally, this would require the use and arrangement of multiple articles, i.e. the headwear or hat, the eyewear and even a strap to secure the eyewear to the head or the headwear. It would be convenient to combine these different articles into a single article of use. Moreover, there may be times when the wearer may wish to don the eyewear while at other times remove the eyewear while still wearing the headwear. In such circumstances it would be advantageous to have ready storage for the eyewear.

The current invention relates to an attachment or kit for releasably securing eyewear to a piece of headwear such as a hat, and more particularly to a connector that allows for the securing of specially constructed lenses or standard eyewear to the headwear. When combined together, two of the connectors and a specially constructed mating lens constitute a kit. Each of the components of the kit releasably attach together and are each replaceable. Further, the connector and the kit may be configured to store within the headwear or hat. The connector may also be configured to removably secure a chin strap to the headwear.

### BRIEF DESCRIPTION OF THE SEVERAL VIEWS OF THE DRAWINGS

The illustrative embodiments of the present invention are shown in the following drawings that form a part of the specification:

FIG. 1 is a perspective view of an embodiment of the eyewear attachment kit of the present disclosure attached to a hat on a wearer's head.

FIG. 2 is a front perspective view an embodiment of the eyewear attachment connector of the present disclosure.

FIG. 3 is a rear perspective view the embodiment of the eyewear attachment connector of the present disclosure depicted in FIG. 2.

FIG. 4 is a side view of the embodiment of the eyewear attachment connector of the present disclosure depicted in FIG. 2.

FIG. 5 is a front view of the embodiment of the eyewear attachment connector of the present disclosure depicted in FIG. 2.

FIG. 6 is a cut-away view of a connector and a lens of an embodiment of the eyewear attachment kit the present disclosure in communion with a sweatband and portion of a hat top or crown.

FIG. 7 is a perspective view of an embodiment of a lens component for an embodiment of the kit of the present disclosure.

FIG. 8 is a perspective view of an embodiment of the eyewear attachment kit of the present disclosure.

FIG. 9 is a perspective view of an alternate embodiment of two of the eyewear attachment connectors of the present disclosure secured to a pair of eyeglasses.

FIG. 10 is a perspective view of an alternate embodiment of two of the eyewear attachment connectors of the present disclosure secured to a pair of eyeglasses.

FIG. 11 is a perspective view of an embodiment of two of the eyewear attachment connectors of the present disclosure, each attached to one end of the same chinstrap.

FIG. 12 is a side view of an alternate embodiment of the eyewear attachment connector of the present disclosure.

FIG. 13 is a front view of the embodiment of the eyewear attachment connector of the present disclosure depicted in FIG. 12.

FIG. 14 is a perspective view of the embodiment of the eyewear attachment connector of the present disclosure depicted in FIG. 12.

FIG. 15 is a perspective view of an alternate embodiment of the eyewear attachment kit of the present disclosure, incorporating the embodiment of the eyewear attachment connector of the present disclosure depicted in FIG. 12, attached to a hat on a wearer's head.

FIG. 16 is a perspective view of yet another alternate embodiment of the eyewear attachment kit of the present disclosure attached to a hat on a wearer's head.

FIG. 17 is a side view of a wearer donning a hat with broken lines depicting an embodiment of the eyewear attachment kit stored inside and positioned generally toward the front portion of the crown of the hat.

FIG. 18 is a side view of a wearer donning a hat with broken lines depicting an embodiment of the eyewear attachment kit stored inside and positioned generally toward the rear portion of the crown of the hat.

Corresponding reference characters indicate corresponding parts throughout the several views of the drawings.

### DETAILED DESCRIPTION OF INVENTION

Multiple configurations of the present invention are contemplated, including various configurations of an eyewear attachment connector and an eyewear attachment kit comprising a pair of connectors. In an illustrative embodiment (FIG. 1), an eyewear attachment kit 10 is shown in conjunction with a hat 12 having a crown 13 and an inner sweatband 14 attached to the bottom edge of the inner surface of the crown 13. The crown 13 has a front portion 15 with a visor or bill 16 extending from the base of the front portion 15, and a rear portion 17. The hat 12 is shown being donned by a wearer 18.

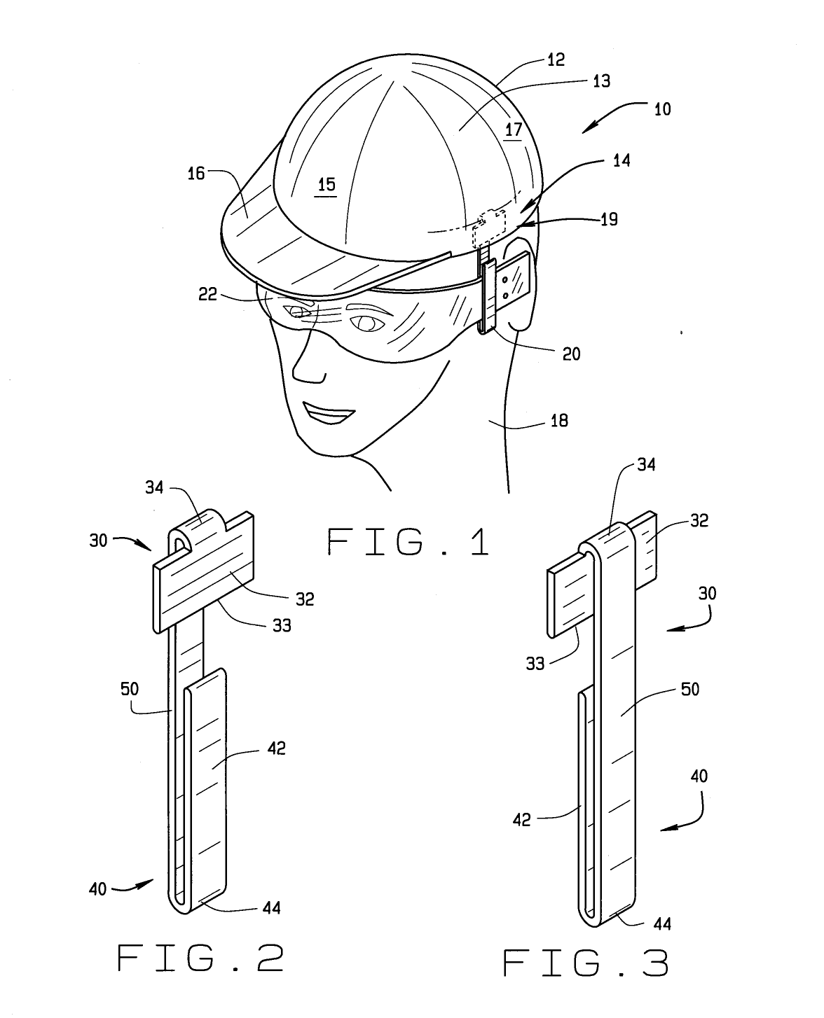

The kit 10 comprises two identical connectors 20 and a lens 22. Shown in more detail and in varying perspectives in FIGS. 2-5, each connector 20 is constructed of a slightly elastic material such as a stiff yet pliant plastic, and comprises an upper portion 30, a lower portion 40, and a generally flat elongated rectangular spine 50 that extends from the top of the upper portion 30 to the bottom of the lower portion 40. The upper portion 30 of the connector 20 comprises a flat rectangular closure pad 32 that is substantially parallel to the spine 50, and extends downward from an apex 34 formed at the juncture between the spine 50 and the pad 32. The pad 32 is wider than the spine 50, forming a horizontal rectangle, with a distal end 33. The apex 34 has a curved construction as the connector 20 is formed of a single piece of plastic. The pliant nature of the material that forms the connector 20 imparts a bias on the pad 32 when the pad is pulled in a direction away from the spine 50. The pad 32 is designed to slip within a standard sweatband 14 between the sweatband 14 and the lower edge of the crown 13 of a hat 14 or similar structure for other forms of headwear. In practice, the pad 32 can be pulled or pried at an angle away from the spine 50 to slip the pad 32 between the sweatband 14 and the base of the headwear or crown 13. The bias caused by separating the pad 32 from the spine 50 will cause the pad 32 to clamp the sweatband 14 between the pad 32 and the spine 50. The length of the pad 32 from the apex 34 is the same as the width of a standard sweatband 14. This insures that when the pad 32 is fully and properly positioned within a standard sweatband 14, the distal end 33 of the pad 32 rests in the bottom of a crease 19 formed at the junction of the bottom of the sweatband 14 and the bottom of the crown 13 and the inner curve of the apex 34 can rest upon the upper edge of the sweatband 14 without causing distortion or wrinkling to the sweatband 14. The width of the pad 32 forms two lever arms about the center of the pad 32 when the pad 32 is positioned within a sweatband 14. As can be appreciated, the distal end 33 of the pad 32 resting in and against the crease 19 provides stability to the connector 20 to minimize forward and rearward rotation of the connector 20 in the sweatband 14. The pad 32 is wider than the spine 50 to establish a longer contact between the distal end 33 of the pad 32 and the crease 19 to provide greater stability to the connector 20.

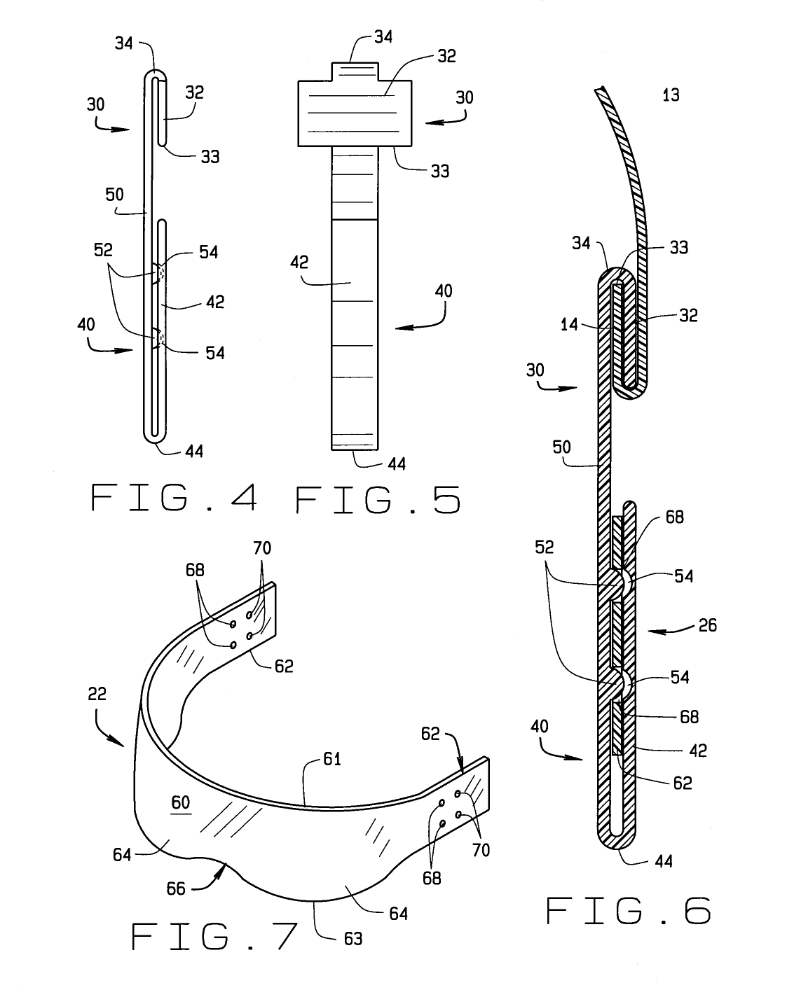

Similar in configuration to the upper portion 30, the lower portion 40 of the connector 20 comprises a flat rectangular closure pad 42 that is substantially parallel to the spine 50, and extends upward from a nadir 44 formed at the junction of the bottom of the spine 50 and the bottom of the pad 42. The pad 42 is generally the same width as the spine 50 and extends upward from the nadir 44 parallel to the spine 50 to just over half the length of the spine 50. The pad 42 and the pad 32 are both positioned on the same side of the spine 50. As can be seen in FIGS. 4 and 6, two semispherical protrusions 52 extend from the surface of the spine 50 facing the pad 42. The protrusions 52 are separated by a distance of approximately one inch along the spine 50 and are each positioned equidistant from both edges of the spine 50. The nadir 44 has a curved construction as the connector 20 is formed of a single piece of plastic. The elastic nature of the material that forms the connector 20 imparts a bias on the pad 42 when the pad is pulled in a direction away from the spine 50. Matching detents 54 in the surface of the pad 42 facing the spine 50 are positioned, sized and shaped to releasably accept the protrusions 52 when the lower portion 40 of the connector 20 is not subject to a bias and is in a relaxed state.

The lens 22 (FIG. 7) is formed of a transparent plastic that is laterally pliant, yet transversely rigid. That is, the lens 22 will readily and resiliently flex and curve with the application of moderate forces perpendicular to the lens faces, while the lens 22 will resist flexing in response to forces applied perpendicular to the edges of the lens. The top 61 of the lens 22 forms a straight line when the lens 22 lies flat. The lens 22 has a central viewing area 60 with a pair of rectangular temples or arms 62 extending laterally in opposing directions from the center of the viewing area 60. The lower edge 63 of the viewing area 60 is shaped to generally conform to the upper portion of a human face in order to form a generic eyewear structure, such that the viewing area 60 has two convex extensions 64 positioned in proximity to a wearer's eyes, and a concave notch 66 positioned in proximity to a wearer's nose, when the lens 22 is bowed into an arch and the kit 10 is worn by the wearer as shown in FIG. 1. Two sequential parallel pairs of grip bores or holes 68 and 70 are formed in the distal end of each of the arms 62. Each of the pairs of holes 68 and 70 are positioned to mate with the matched protrusions 52 and detents 54 in the lower portions 40 of the connectors 20. The holes 68 and 70 have a diameter that is just slightly larger than the diameter of the protrusions 52, such that the protrusions 52 fit snugly within but can be readily removed from the holes 68 and 70.

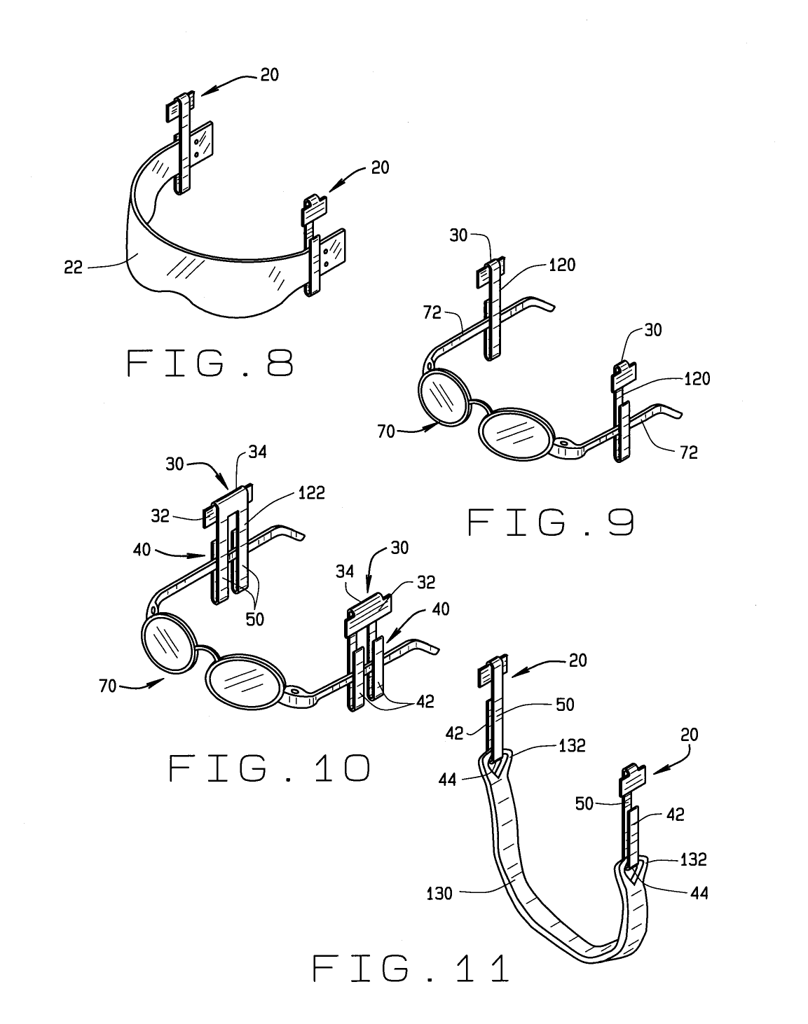

As can be appreciated, the configuration of the lower portions 40 of each of the connectors 20, and in particular the fit of the holes 68 and 70 to the matched protrusions 52 and detents 54, is such that when a connector 20 is releasably attached to each of the arms 62 with the protrusions 52 properly positioned within one of the pairs of holes 68 or 70 of the respective connector 20, the connectors 20 are thereupon capable of holding the lens 22 in a cantilever fashion as illustrated in FIG. 8. When the lower portions 40 of the connectors 20 are thus attached to a lens 22, and when (FIG. 1) the upper portions 30 of both connectors 20 are attached to and hanging from a sweatband 14 in a piece of headwear such as for example, a hat 12, where the connectors 20 are positioned such that the lens 22 extends in a curve about the front of the headwear or hat 12 below the bill 16, the connectors 20 thereby support the lens 22 in a cantilever fashion in front of the eyes of a wearer 18 donning the headwear or hat 12. Of course, the materials forming the connectors 20 must be of sufficient rigidity and the connection between the connectors 20 and the lens 22 must be sufficiently secure to prevent any significant bending or twisting that would allow the lens 22 to droop or sag from a generally perpendicular cantilevered position below the bill 16 of the headwear or hat 12. While it would be possible to produce connectors 20 or a kit 10 with insufficient rigidity or strength to independently support the lens 22 in a cantilever fashion below the bill 16 of a hat 12 or other headwear, such that the lens 22 would require support from the wearer's face or from some other source, the disclosed embodiments of the connectors 20 and the kit 10 require no such supplemental support.

As can also be appreciated, the forward positioning of the lens 22 below the bill 16 is readily adjustable relative to the headwear or hat 12. First, each connector 20 may be positioned along the sweatband 14 forward or rearward to suit the specific comfort or tastes of each individual wearer 14. Moreover, each arm 62 of the lens 22 is configured with two pairs of grip holes 68 and 70, where the grip holes 68 are positioned closer to the viewing area 60 than the grip holes 70. Thus, the grip holes 68 and 70 allow the lens 22 to be connected to each connector 20 in more than one position along the length of the arms 62 for even more adjustability.

Additional embodiments of the current invention are depicted in FIGS. 9-13. In one such embodiment, FIG. 9 discloses a pair of alternate connectors 120 configured to support eyewear 70 having a pair of temples or arms 72, such as a pair of spectacles or eyeglasses, in place of the lens 22. Similar to the embodiment of FIGS. 1-8, the eyewear 70 is suspended from a piece of headwear such as a hat 12, when the upper portions 30 of both connectors 120 are attached to and hanging from a sweatband 14 in a the headwear, and where the connectors 120 are positioned along the sweatband 14 and connected to the arms 72 of the eyewear, such that the lenses of the eyewear 70 are positioned below the bill 16 of the hat 12. Because this configuration lacks the holes 68 and 70 of the lens 22, the protrusions 52 in the spines 50 and the detents 54 in the pads 42 are shaped and sized to snap together to releasably attach the pads 42 to the respective spines 50 and thereby secure the arms 72 to the connectors 120. It will be appreciated that the material forming the connectors 120 in this configuration may be somewhat more pliant than in other configurations so as to allow the pad 42 or the spine 50 of each connector 20 to flex about the arms 72 for a snug fit. Alternately, a channel may be formed in the pad 42 or the spine 50 in which the arms 72 may be positioned when the connectors 120 are secured to the arms 72. The connectors 120 thereby support the eyewear 70 in a cantilever fashion in front of the eyes of a wearer 18 donning the headwear or hat 12. Of course, for some models, the weight of the eyewear 70 may be such that the eyewear 70 will yet twist downward at the connection between the arms 72 and the connectors 120, and the connectors 120 as depicted in FIG. 9 would not have sufficient strength to maintain the eyewear 70 in a cantilevered position. One solution to this circumstance would be to merely set the eyewear 70 on the bridge of the nose of the wearer 18. Another solution is found in the embodiment of the connector 20 disclosed in FIG. 10.

In FIG. 10, alternate connectors 122 are each configured with a single upper portion 30, but with a pair of matching laterally spaced lower portions 40. Each of the matching lower portions 40 has its own spine 50 that attaches to the pad 32 of the upper portion 30 at the apex 34. The pad 32 may be wider in this embodiment than in the embodiments of FIGS. 1-11 to provide more rotational stability to the connectors 122 when in the sweatband 14 of a piece of headwear. As shown in FIG. 10, two of the alternate connectors 122 are configured to support eyewear 70 having a pair of temples or arms 72, such as a pair of spectacles or eyeglasses. Again, the eyewear 70 is suspended from a piece of headwear such as a hat 12, when the upper portions 30 of both connectors 122 are attached to and hanging from a sweatband 14 in a the headwear, and where the connectors 20 are positioned along the sweatband 14 and connected to the arms 72 of the eyewear, such that the lenses of the eyewear 70 are positioned below the bill 16 of the hat 12. Because this configuration also lacks the holes 68 and 70 of the lens 22, the protrusions 52 in the spines 50 and the detents 54 in the pads 42 are shaped and sized to snap together to releasably attach the pads 42 to the respective spines 50 and thereby secure the arms 72 to the connectors 122.

As shown in FIG. 11, a chinstrap 130 may be releasably attached to the connectors 20. The chinstrap 130 may be configured with a loop 132 at each end. Each loop 132 can be positioned in the nadir 44 of each connector 20 between the pad 42 and the spine 50. It will be appreciated that each of the embodiments of the connectors 20, 120 and 122 of the FIGS. 1-10 are each adaptable to receive and hold a chinstrap such as for example the chinstrap 132. Of course, the chinstrap 130 can be provided as an independent item or packaged as part of an expanded kit 10.

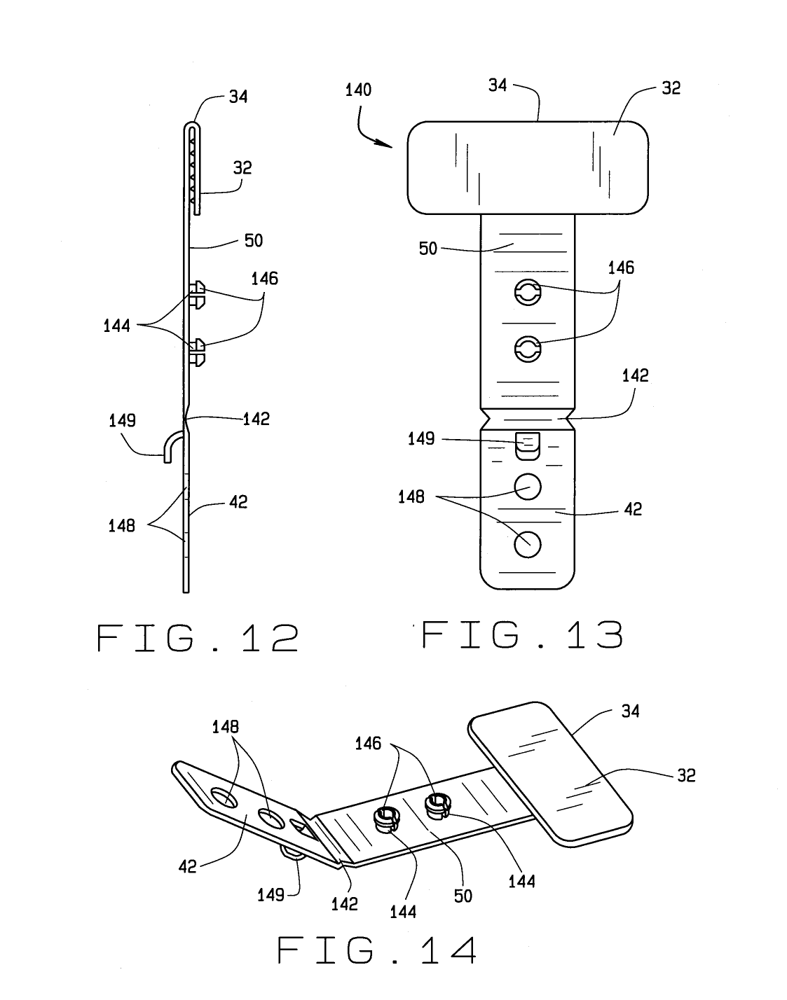

In yet another illustrative embodiment of the present invention, FIGS. 12-14 show a connector 140 having a transverse living hinge 142 located at the junction of the spine 50 and the pad 42. The connector 140 lacks protrusions 52 and detents 54. Rather, a pair of split elongated cylindrical prongs 144, having split slightly broadened cylindrical or semispherical heads 146, extend from the surface of the spine 50 facing the pad 42. The prongs 144 are separated by a distance of approximately one inch along the spine 50 and are each positioned equidistant from both edges of the spine 50. Matching bores or holes 148 in the surface of the pad 42 facing the spine 50 are positioned, sized and shaped to releasably accept the prongs 144 when the pad 42 is pressed against the spine 50. The prongs 144 and holes 148 are configured to match the positioning of the holes 68 and 70 of the lens 22, and the prongs 144 are sized to fit through the holes 68 and 70. The prongs 144 are just long enough to extend through the holes 68 or 70 of through the lens 22, through the holes 148 in the pad 42, and allow the heads 146 to protrude fully through to the other side of the pad 42, where the heads 146 will expand to snap in place and releasably hold the pad 42 and the lens 22 to the spine 50. That is, the split heads 146 have a diameter that is slightly greater than the diameter of the holes 148, such that with a reasonable amount of force the heads 146 can be pushed through the holes 148 and expand to hold the pad 42 and the lens 22 to the spine 50, in a snap-like fashion. In addition, the connector 140 has a curved chin strap hook 149 protruding upwardly from the surface of the pad 42 opposite the spine 50. The hook 149 extends outward away from the spine 50 when the pad 42 is near or snapped to the spine 50. As can be appreciated, the hook 149 allows for the removable attachment of a chin strap (not shown), such as for example a looped chin strap 130 as shown in FIG. 11, to the connector 140 by placing the loop 132 over the hook 149. Alternately, a chin strap can be tied to the hook 149 or otherwise removably secured to the hook 149. This hook feature 148 enables the connector 140 to provide the dual function of removably securing a lens 22 or other suitable eyewear to a piece of headwear such as a hat 12, while also allowing a chinstrap to be removably secured to the same piece of headwear or hat 12.

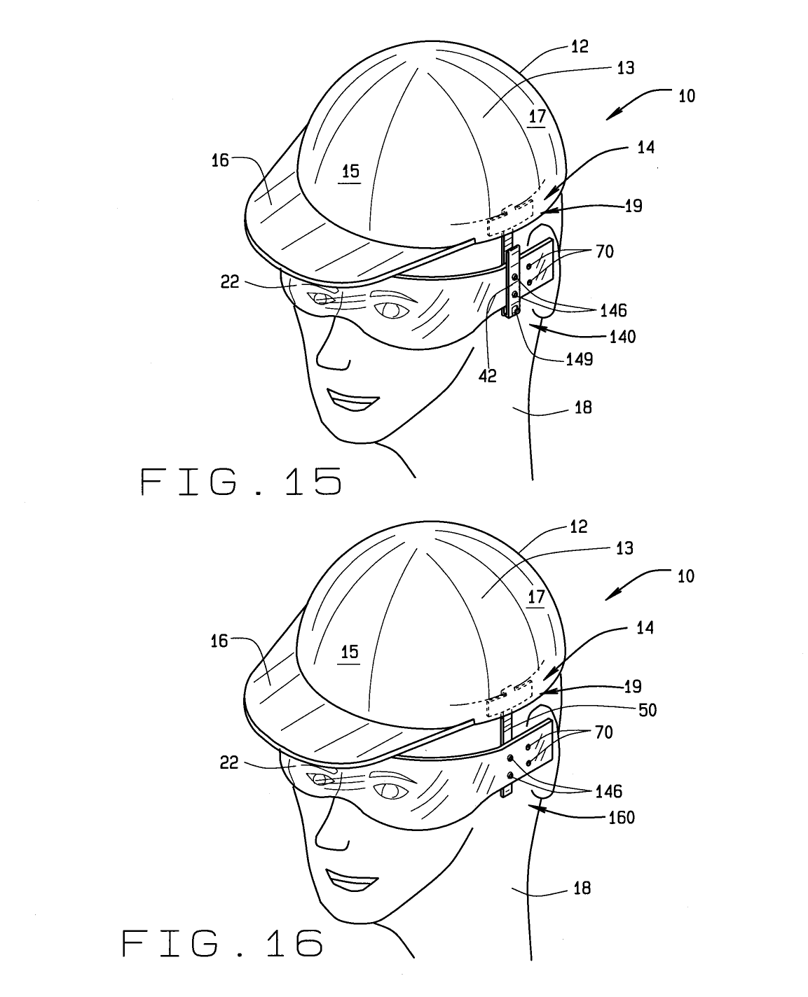

FIG. 15 shows an embodiment of the kit 10 releasably attached to a hat 12 that has been donned by a wearer, in which the kit 10 incorporates two of the alternate connectors 140 in place of the connectors 40. As can be appreciated, the lens 22 is held in rigid, yet releasable, attachment to the connector 140 when the prongs 144 (not shown) and heads 146 are pushed through the holes 148 and expand to hold the pad 42 and the lens 22 to the spine 50, in a snap-like fashion.

FIG. 16 shows yet another embodiment of the kit 10 which comprises a pair of alternate connectors 160. Each connector 160 comprises the same features and components as the connector 140, but lacks the hinge 142 and the pad 42. Lacking the pad 42, the connector also lacks the holes 148 and the hook 149. However, a similar hook that is capable of attachment to a chinstrap may be formed on or attached to the spine 50 of the connector 160. In this embodiment of the kit 10, the prongs 144 and holes 148 of the connector 160 are configured to match the positioning of the holes 68 and 70 of the lens 22, and the prongs 144 are sized to fit through the holes 68 and 70. The prongs 144 are just long enough to extend through the holes 68 or 70 of through the lens 22 and allow the heads 146 to protrude fully through to the other side of the lens 22, where the heads 146 will expand to snap in place and releasably hold the lens 22 to the spine 50. That is, the split heads 146 have a diameter that is slightly greater than the diameter of the holes 68 and 70, such that with a reasonable amount of force the heads 146 can be pushed through the holes 68 or 70 and expand to hold the lens 22 to the spine 50, in a snap-like fashion. Of course, the holes 68 and 70 or, alternately, the prongs 144 and heads 146, can be adjusted in size and shape to facilitate this snap-like connection.

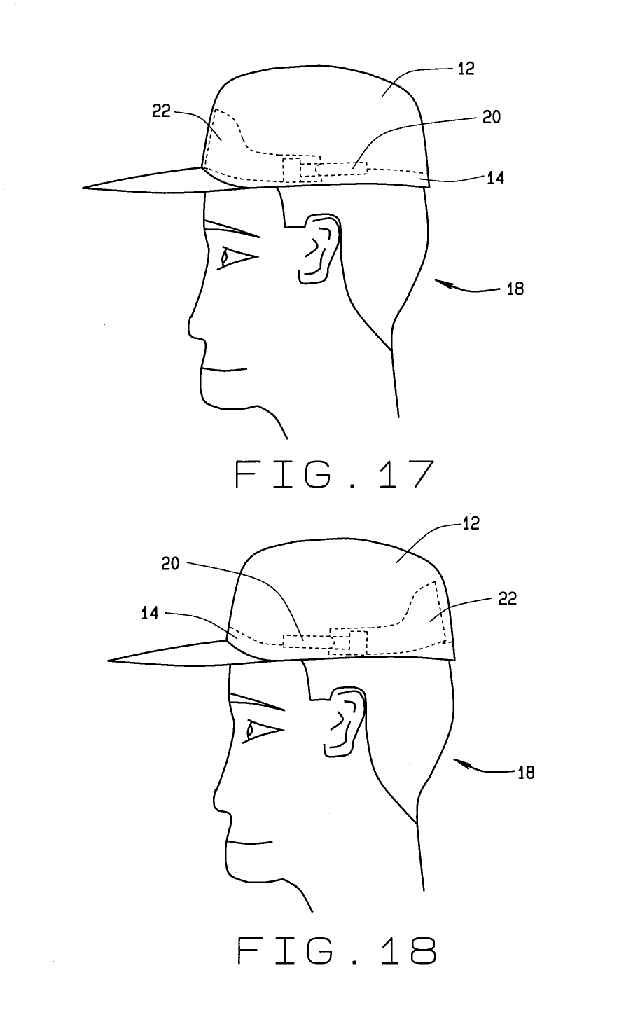

FIGS. 17 and 18 disclose yet another unique feature of various embodiments of the current invention. Each of the connectors 20, 120, 122 and 140 are configured to slip between the sweatband 14 and the lower portion of the headwear or hat 12 as shown in FIGS. 17 and 18. In FIG. 17, a connector 20 is shown positioned on the side of the head of a wearer 18 within the hat 12, with a lens 22 positioned towards the front of the hat 12. In FIG. 18 a connector 20 is shown positioned on the side of the head of a wearer 18 within the hat 12, with a lens 22 positioned towards the rear of the hat 12. As can be appreciated, the flexible nature of the lens 22 and the thin and somewhat pliant construction of the connectors 20, 120, 122 and 140, allow for the individual or joint storage of each of these components of the kit 10 secured by the sweatband 14 at virtually any position along the sweatband 14 within a piece of headwear of a hat 12. Thus, a kit 10, or components of a kit 10 can be secured or stored within a piece of headwear, such as for example a hat 12, and can readily be removed from such storage at any time for rapid positioning on the headwear for use. A clip or other similar structure or feature (not shown) may be constructed along any edge of any of the connectors 20, 120, 122 or 140, to secure the connector in a horizontal position in the sweatband 14 (as shown in FIGS. 17 and 18) for storage.

Variations exist with respect to the inventive connectors 20, 120, 122 and 140, and kit 10. For example, the apex 34 may be other constructs, such as for example, a seam, a hinge, or a ridge, depending upon various factors, such as the desired flexibility at the apex 34 and the manufacturing method employed to fabricate the connector 20. Further, the pads 32 and 42, and the spine 50, may each comprise a variety of shapes other than a rectangle, such as for example, ovoid or polygonal, and each may be of different thicknesses, so long as each performs its designated function as disclosed herein. Further, the pads 32 and 42 need not be positioned on the same side of the spine 50. The protrusions 52 may be of a variety of shapes, lengths and sizes, so long as the detents 54 match the protrusions 52 and the protrusions 52 and detents 54 provide the support to the lens 22 as disclosed. Further, there may be more than two protrusions 52 and matching detents 54 in each connector 20, 120 and 122, and the protrusions 52 and matching detents 54 may be positioned in different locations than shown in the depicted embodiments. Similarly, for the connector 140, there may be more than two prongs 144 and matching holes 148 in each connector, and the prongs 144 and matching holes 148 may be positioned in different locations than shown in the depicted embodiment.

Of course, the present invention may also be used with other forms of headwear other than a hat such as at 12, so long as the headwear comprises a sweatband such as at 14, a portion of a sweat band, or other such feature or component that allows the connector to releasably attach to and suspend from the headwear.

Regarding the kit 10, the lens 22 can be for example constructed of more rigid materials, or framed in more rigid materials, such that the lens maintains a designated form without the ability to flex. The viewing area 60 may be of many shapes and sizes to provide a wide variety and styles of eyewear. For example, the viewing area 60 may have no extensions 64 and a small notch 66, or no notch 66 at all. Alternately, the viewing area 60 may have parallel top and bottom edges. Further, the lens 22 is not required to have exactly two pairs of holes 68 and 70 to mate with the protrusions 52 and detents 54 in the lower portions 40 of the connectors 20. Rather, the lens 22 may have for example one set of holes 68 or 70 in each arm 62. Alternately, each arm 62 may have more than two pairs of holes, such as 68 or 70, so long as each pair of holes is configured and positioned to mate with the connectors' protrusions 52 and detents 54. In yet another variation, the lens 22 need not have any holes or bores 68, 70, but the cantilever support from the connectors 20 may be provided by other well understood means such as, for example, compressive pressure between the spine 50 and the pad 42. In another example, the lens 22 may be formed with protrusions or other features that releasably attach to the connector 20 to provide a sufficiently rigid connection to provide cantilever support for the lens 22, with no need for the bottom pad 42 or 142.

Further variations on the basic construction are also available. For example, the exact shape and size of each of the connectors 20, 120, 122 and 140, and of each of the components of the kit 10 can be varied to form larger or smaller configurations, or to form shapes and sizes for specific applications. Each of the connectors 20, 120, 122 and 140 may be constructed of a variety of materials or combinations of materials, including but not limited to metal, rubber, plastic and foam, or any other suitable material that allows each connector to properly operate as disclosed. Likewise, each of the components of the kit 10 may be constructed of a variety of materials, including but not limited to metal, rubber, plastic and foam, or any other suitable material that allows each component to perform its function within the kit 10. Each dimension of the connectors 20, 120, 122 and 140 and of the kit 10, and thereby all of the kit's components, may be of varying sizes, so long as the connectors and components perform their desired functions as disclosed.

The detailed description above illustrates the invention by way of example and not by way of limitation. This description clearly enables one skilled in the art to make and use the invention, and describes several embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

1. An eyewear attachment connector comprising an upper portion and a lower portion and a spine there between, the upper end of the spine having an apex at its upper end and a nadir at its lower end, the upper portion configured to releasably attach the connector to and suspend the connector from a sweatband positioned horizontally within a piece of headwear, the lower portion configured to releasably hold a first arm of a piece of eyewear in a substantially horizontal orientation relative to the sweatband when said first arm is held between the lower portion and the spine and when the upper portion is suspended from the sweatband.

2. The connector of claim 1, wherein the upper portion comprises an upper pad attached at the apex of the spine, the upper pad configured to form an upper gap between the upper pad and the spine for the positioning of the sweatband in the upper gap thereby to releasably attach the connector to the sweatband, the gap being slightly thinner than the thickness of the sweatband.

3. The connector of claim 2, wherein the connector is comprised at least in part of a resilient material such that increasing the width of the upper gap creates a bias that causes the upper pad and spine to squeeze together against the sweatband when the sweatband is forced there between.

4. The connector of claim 1, wherein the lower portion comprises a lower pad attached at the nadir of the spine, the lower pad configured to form a lower gap between the lower pad and the spine for the positioning of the first arm of a piece of eyewear in the lower gap to thereby releasably hold the first arm.

5. The connector of claim 4, wherein the connector is comprised at least in part of a resilient material such that increasing the width of the lower gap creates a bias that causes the lower pad and spine to squeeze together against the first arm of the eyewear when the first arm is forced there between.

6. The connector of claim 1, further comprising a hinge positioned along the nadir of the spine to rotationally connect the lower pad to the spine.

7. The connector of claim 6, wherein the hinge is a living hinge.

8. The connector of claim 4, further comprising a prong and a hole, the hole being formed in one of said lower pad and spine and the prong extending from the other of said lower pad and spine toward the hole, the hole being adapted to snugly accept the prong in the hole when the lower pad and spine are forced together.

9. The connector of claim 8, further comprising a head on the end of the prong, the head being slightly larger than the hole and sufficiently pliant to allow the head to at least in part squeeze through the hole when the lower pad and spine are forced together to snap the lower pad to the spine.

10. The connector of claim 1, wherein the lower portion is configured to hold the first arm of a specific configuration of eyewear.

11. The connector of claim 1, further comprising a protrusion and a detent, the detent being formed in one of said lower pad and spine and the protrusion extending from the other of said lower pad and spine toward the detent, the detent being positioned and adapted to accept the protrusion in the detent when the lower pad and spine are directed together.

12. The connector of claim 11 in combination with a piece of eyewear having a detent along its first arm, wherein the protrusion is positioned on the connector to mate with the detent on the arm such that the lower portion releasably holds the first arm in a substantially horizontal orientation relative to the sweatband when said first arm is positioned between the lower portion and the spine and when the upper portion is attached to the sweatband within the headwear.

13. The connector of claim 1, wherein the headwear is a hat and the connector is configured to store inside the hat, such that no portion of the connector is visible outside the hat when the hat is worn.

14. The connector of claim 13, wherein the connector is configured to store, at least in part, between the sweatband and the inner surface of the hat.

15. The connector of claim 1, further comprising a hook adapted to releasably hold a chinstrap to the connector.

16. An eyewear kit adapted for releasable attachment to a piece of headwear comprising a sweatband positioned horizontally within the headwear, the sweatband having a left side and a right side, the kit comprising:
a. a lens configured to cover at least in part one eye of a wearer donning the headwear, the lens having a left arm and a right arm; and
b. first and second connectors, each connector comprising an upper portion and a lower portion, the upper portion configured to releasably attach the first connector to the sweatband such that the connector is suspended from the sweatband, the lower portion configured to releasably hold one of said lens arms in an orientation below and substantially parallel to the orientation of the sweatband when the upper portion is attached to the sweatband; and
wherein when the upper portion of one of said connectors is attached to the left side of the sweatband and the lower portion of the same connector is holding the left arm of the lens, and the upper portion of the other connector is attached to the right side of the sweatband and the lower portion of the other connector is holding the right arm of the lens, the connectors suspend the lens in a position at least in part over one of the wearer's eyes when the headwear is donned by the wearer such that the left side of the sweatband is oriented substantially above the wearer's left temple and the right side of the sweatband is oriented substantially above the wearer's right temple.

17. The kit of claim 16, wherein the first and second connectors are adapted to removably attach to a chinstrap.

18. The kit of claim 17, further comprising a chinstrap attached to the first and second connectors.

19. The kit of claim 16, wherein the lens is laterally pliant.

20. The kit of claim 16, wherein the lens comprises two portions, each portion configured to cover one of the wearer's eyes.

21. The kit of claim 16, wherein the lens is positioned over both of the wearer's eyes when the headwear is donned by the wearer.

22. The kit of claim 16, wherein each arm of the lens comprises a bore adapted to mate with a projection on the lower portion of one of said connectors.

23. The kit of claim 16, wherein the headwear is a hat and the connectors are configured to store inside the hat, such that no portion of the connectors are visible outside the hat when the hat is worn.

24. The connector of claim 23, wherein the connectors are configured to store, at least in part, between the sweatband and the inner surface of the hat.

25. The kit of claim 16, wherein the lens is configured to store inside the hat, such that no portion of the lens is viewable when the hat is worn.

26. The kit of claim 16, further comprising a hook adapted to releasably hold a chinstrap to the connector.

27. The kit of claim 16, wherein the connectors are configured to be interchangeable with each other.

28. The kit of claim 16, further comprising a container to house the kit.
